(19) United States
(12) Patent Application Publication
(10) Pub. No.: US 2025/0311796 A1
(43) Pub. Date: Oct. 9, 2025

(54) Catheter Undergarment Device

(71) Applicants: Lloyd Jackson, SR., Bakersfield, CA (US); Sonja Smith Williams, Las Vegas, NV (US); Delora Jackson, Bakersfield, CA (US)

(72) Inventors: Lloyd Jackson, SR., Bakersfield, CA (US); Sonja Smith Williams, Las Vegas, NV (US); Delora Jackson, Bakersfield, CA (US)

(21) Appl. No.: 19/041,345

(22) Filed: Jan. 30, 2025

Related U.S. Application Data
(60) Provisional application No. 63/631,493, filed on Apr. 9, 2024.

(51) Int. Cl. A41D 13/12; A41B 9/00; A41B 9/14; A41D 27/20; A61F 13/49

(52) U.S. Cl. CPC A41D 13/1254; A41B 9/001; A41B 9/14; A41D 27/20; A41B 2400/34; A61F 13/49

(57) ABSTRACT

A catheter undergarment device is disclosed, which is an undergarment designed to enhance comfort and discretion for individuals using catheters. The catheter undergarment device comprises a body component configured as a diaper or adult undergarment with a catheter opening that ensures both the catheter and catheter bag remain concealed beneath clothing. The body component is constructed using a waterproof material to catch spills, is non-chafing, and fits snugly like regular underwear. Antibiotic ointment will be around the fitting of the catheter opening in order to help treat or stop any infections from happening. The device allows users to engage comfortably in daily activities and wear a variety of clothing, including casual or professional looking clothing, without visibility of the catheter apparatus.

### CROSS-REFERENCE TO RELATED APPLICATION

The present application claims priority to, and the benefit of, U.S. Provisional Application No. 63/631,493, which was filed on Apr. 9, 2024, and is incorporated herein by reference in its entirety.

### FIELD OF THE INVENTION

The present invention relates generally to the field of catheter undergarment devices. More specifically, the present invention relates to undergarments designed for people wearing catheters daily for long or short term time periods. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in catheter undergarment devices. Generally, people who wear catheters daily may struggle with keeping the catheter bag hidden. As such, significant embarrassment can occur if the bag is visible, especially if the bag is full. Further, any accidental catheter leaks will inevitably stain clothing, causing even more embarrassment and requiring cleaning.

Additionally, catheter tubing is commonly used in medical settings such as in hospitals and hospices. Often such tubing enters or exits the body of the patient in or around the groin. Traditionally, the catheter tubing is merely threaded through leg openings, such as the leg opening of a diaper. Such positioning can lead to increased discomfort for the patient by increasing the risk for leakage, skin breakdown, and occlusion. Additionally, the pressure caused by the catheter tubing being pressed down as it exits the legging can increase pain and risk of displacement.

Accordingly, there is a demand for an improved catheter undergarment device that improves comfort and self-confidence of catheter users. More particularly, there is a demand for a catheter undergarment device that discloses an improved undergarment designed for people wearing catheters daily for a period of time.

Therefore, there exists a long-felt need in the art for a catheter undergarment device that provides users with an improved undergarment designed for people wearing catheters daily for long or short term time periods. There is also a long-felt need in the art for a catheter undergarment device that features a specialized catheter opening that perfectly accommodates the device while ensuring it remains not visible in any way. Further, there is a long-felt need in the art for a catheter undergarment device that allows users to discreetly hide their catheter bags without worry of it being exposed to the public eye. Moreover, there is a long-felt need in the art for a device that enables wearers to comfortably engage in daily activities without worry of spills, drippings, or any irritation or rash. Further, there is a long-felt need in the art for a catheter undergarment device that improves comfort and self-confidence for any male or female wearing a catheter. Finally, there is a long-felt need in the art for a catheter undergarment device that includes an antibiotic ointment around the fitting of the catheter slot.

The subject matter disclosed and claimed herein, in one embodiment thereof, comprises a catheter undergarment device. The device is an undergarment designed to enhance comfort and discretion for individuals using catheters. The catheter undergarment device comprises a body component configured as a diaper or adult undergarment with a catheter opening that ensures both the catheter and catheter bag remain concealed beneath clothing. The body component is constructed using a waterproof material to catch spills, is non-chafing, and fits snugly like regular underwear. Antibiotic ointment will be around the fitting of the catheter opening in order to help treat or stop any infections from happening. The device allows users to engage comfortably in daily activities and wear a variety of clothing, including casual or professional looking clothing, without visibility of the catheter apparatus.

In this manner, the catheter undergarment device of the present invention accomplishes all of the foregoing objectives and provides users with a device that allows users to discreetly hide their catheter bags without worry of spills. The device is an undergarment with a catheter opening and means for securely retaining a catheter bag. The device is manufactured of waterproof materials.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a catheter undergarment device. The device is an undergarment designed to enhance comfort and discretion for individuals using catheters. The catheter undergarment device comprises a body component configured as a diaper or adult undergarment with a catheter opening that ensures both the catheter and catheter bag remain concealed beneath clothing. The device allows users to engage comfortably in daily activities and wear a variety of clothing, including casual or professional looking clothing, without visibility of the catheter apparatus.

In one embodiment, the catheter undergarment allows users to discreetly hide their catheter bags without worry of it being exposed to the public eye. Further, the device enables users to comfortably engage in daily activities without worry of spills, drippings, or any irritation or rash, etc. Additionally, the catheter undergarment device improves comfort and self-confidence for any male or female wearing a catheter.

In one embodiment, the catheter undergarment comprises a body component configured as a diaper or adult undergarment. The body component is constructed using a waterproof material to catch spills, is non-chafing, and fits snugly like regular underwear. The body component comprises a rear portion, a front panel extending from the rear portion, and opposing side panels. The rear portion is adapted to be positioned underneath the buttocks of a user, with the opposing side panels being secured around the legs of the user before connecting to the front panel so as to form a unified undergarment configuration. Typically, the rear portion and front panel are integrally formed together. Further, the opposing side panels are releasably secured to each other via hook and loop fasteners or tabs (i.e., Velcro), clasps, buttons, bands, adhesives, straps, snaps, etc. The use of side panels adapted to be removably secured together aids in applying or removing the undergarment (i.e., body component) on immobile patients, such as those who are bedridden.

In one embodiment, the upper end of the front panel and the rear portion forms the waistband of the undergarment (i.e., body component) and is adapted to encircle the user's waist. The lower end of the front panel and the rear portion includes a curved portion on the sides to accommodate leg openings when the undergarment (i.e. body component) is formed.

In one embodiment, the rear portion, front panel, and opposing side panels are all integrally formed together as one integral piece (i.e., unitary structure).

In one embodiment, the body component comprises a flexible, waterproof material. Further, the body component is preferably at least partially comprised of a material and configuration which will act as an absorbent for body fluids and the like, such as those resulting from urination or defecation.

In one embodiment, the front panel and rear portion both include an outer surface and an inner surface. The inner surface contacts a user's skin and is at least partially comprised of an absorptive material for absorbing liquids, feces, and such. The outer surface contacts a user's outer clothing and is waterproof, so as to prevent leaks on the outer clothing, etc.

In one embodiment, the front panel comprises a catheter opening. The catheter opening is a through-hole through the front panel for accepting a catheter tube. Specifically, the catheter opening is a through-hole through which a catheter tube may be extended. A tube retainer may also be utilized to guide the catheter tube out of the body component through the catheter opening. The catheter opening is adapted to allow a catheter tube or other medical instrument or device to pass through the catheter opening. The present invention is preferably utilized with an indwelling catheter which comprises a catheter tube having an end inserted subcutaneously. The distal end of the catheter tube extends through the catheter opening and, in some embodiments, a tube retainer helps to guide the tube through the opening. The tube retainer lines the bottom of the opening and may be utilized to guide the catheter tube as it exits the catheter opening. The catheter tube may extend through any portion of the catheter opening.

In one embodiment, the catheter opening comprises a pad which is adapted to be positioned behind the catheter opening between the body of the user and the undergarment (i.e., body component). The pad is generally comprised of a piece of fabric or other absorbent material which is adapted to be positioned behind the catheter opening and around the catheter tube. The pad acts to prevent leaks, drips, or spills from the catheter tube.

In one embodiment, the catheter opening comprises antibiotic ointment around the perimeter of the opening. Any suitable antibiotic ointment can be utilized as is known in the art. The antibiotic ointment helps treat or stop any infections from happening.

In one embodiment, the catheter undergarment device comprises leg straps that secure the catheter bag to a user's inner leg. The leg straps typically are secured via Velcro, snaps, adhesives, buckles, etc. In another embodiment, the catheter undergarment includes an inner leg pouch on one leg that is secured via leg straps around a user's leg. The leg straps typically are secured via Velcro, snaps, adhesives, buckles, etc. The tube and catheter bag are hidden within the inner leg pouch. The inner leg pouch is constructed to expand and it allows for the catheter bag to expand when retaining urine collection. The leg straps for either embodiment can be knee length, calf length, or ankle length and made of an elastic and expandable material.

In one embodiment, the side panels of the body component can be extended down, creating a leg component, such that the body component is configured as a pair of shorts. In this embodiment, the leg component comprises a pocket which can retain the catheter bag, such that the catheter bag does not have to be secured with straps to a leg. Specifically, leg straps can irritate skin from the leg straps constantly sliding up and down legs. To eliminate this movement, users typically would overtighten their leg straps, which in turn would reduce blood flood to their leg, further complicating an already bad situation. Users also inadvertently dislodge tubes while trying to undress or go to the restroom.

In one embodiment, to keep the urine discharge from moving up in the tubing, a one-way valve is recommended. The one-way valve can be pressure sensitive and can be positioned in the catheter tube or at the entry into the catheter bag.

In one embodiment, the catheter undergarment device is manufactured from waterproof material to catch spills, is non-chafing, and fits snugly like regular underwear, or other suitable materials as is known in the art, or a combination of such materials. Further, the device is manufactured of a stretchable, elastic-type fabric that may include fabric blends such as cotton/poly/Spandexe or Nylon® Tactel® & LYCRA®, etc.

In yet another embodiment, the catheter undergarment device comprises a plurality of indicia.

In yet another embodiment, a method of allowing users to discreetly hide their catheter bags without worry of spills is disclosed. The method includes the steps of providing a catheter undergarment device comprising a body component with a catheter opening. The method also comprises wearing the body component as an undergarment. Further, the method comprises threading the catheter tubing through the catheter opening. The method also comprises securing the catheter bag to a user's leg via straps. Finally, the method comprises using the device concealed beneath clothing.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of the catheter undergarment device of the present invention in accordance with the disclosed architecture;

FIG. 2 illustrates a perspective view of one embodiment of the catheter undergarment device of the present invention showing the catheter bag secured to a leg in accordance with the disclosed architecture;

FIG. 3 illustrates a perspective view of one embodiment of the catheter undergarment device of the present invention showing the catheter undergarment on a user in accordance with the disclosed architecture;

FIG. 4 illustrates a perspective view of one embodiment of the catheter undergarment device of the present invention showing the catheter opening in accordance with the disclosed architecture;

FIG. 5 illustrates a perspective view of one embodiment of the catheter undergarment device of the present invention showing the device in use in accordance with the disclosed architecture; and

FIG. 6 illustrates a flowchart showing the method of allowing users to discreetly hide their catheter bags without worry of spills in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a catheter undergarment device that provides users with an improved undergarment designed for people wearing catheters daily for long or short term time periods. There is also a long-felt need in the art for a catheter undergarment device that features a specialized catheter opening that perfectly accommodates the device while ensuring it remains not visible in any way. Further, there is a long-felt need in the art for a catheter undergarment device that allows users to discreetly hide their catheter bags without worry of it being exposed to the public eye. Moreover, there is a long-felt need in the art for a device that enables wearers to comfortably engage in daily activities without worry of spills, drippings, or any irritation or rash. Further, there is a long-felt need in the art for a catheter undergarment device that improves comfort and self-confidence for any male or female wearing a catheter. Finally, there is a long-felt need in the art for a catheter undergarment device that includes an antibiotic ointment around the fitting of the catheter slot.

The present invention, in one exemplary embodiment, is a novel catheter undergarment device. The catheter undergarment device comprises a body component configured as a diaper or adult undergarment with a catheter opening that ensures both the catheter and catheter bag remain concealed beneath clothing. The body component is constructed using a waterproof material to catch spills, is non-chafing, and fits snugly like regular underwear. Antibiotic ointment will be around the fitting of the catheter opening in order to help treat or stop any infections from happening. The device allows users to engage comfortably in daily activities and wear a variety of clothing, including casual or professional looking clothing, without visibility of the catheter apparatus. The present invention also includes a novel method of allowing users to discreetly hide their catheter bags without worry of spills. The method includes the steps of providing a catheter undergarment device comprising a body component with a catheter opening. The method also comprises wearing the body component as an undergarment. Further, the method comprises threading the catheter tubing through the catheter opening. The method also comprises securing the catheter bag to a user's leg via straps. Finally, the method comprises using the device concealed beneath clothing.

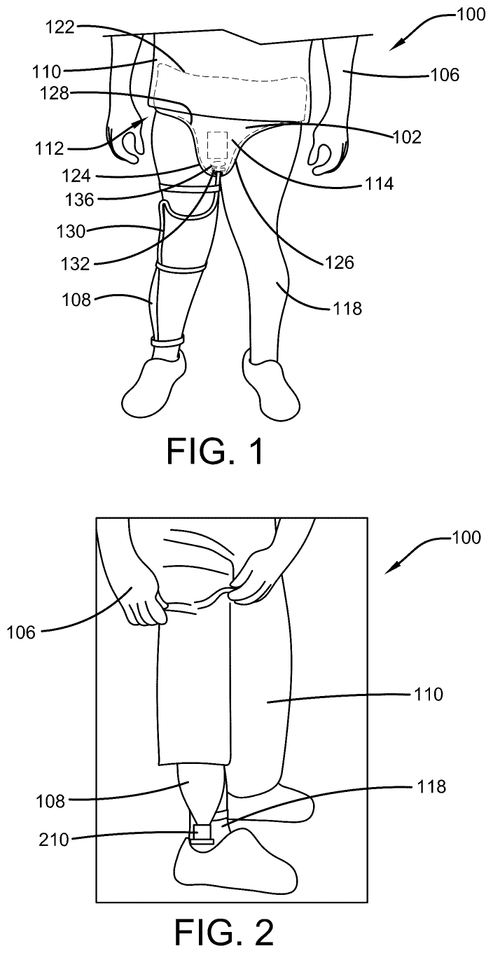

Referring initially to the drawings, FIG. 1 illustrates a perspective view of one embodiment of the catheter undergarment device 100 of the present invention. In the present embodiment, the catheter undergarment device 100 is an improved undergarment that provides a user 106 with a means for concealing catheter bags 108. Specifically, the catheter undergarment device 100 comprises a body component 102 and a catheter opening 104. The device 100 allows users 106 to engage comfortably in daily activities and wear a variety of clothing 110, including casual or professional looking clothing 110, without visibility of the catheter apparatus.

Generally, the catheter undergarment device 100 allows users 106 to discreetly hide their catheter bags 108 without worry of it being exposed to the public eye. Further, the device 100 enables users 106 to comfortably engage in daily activities without worry of spills, drippings, any irritation or rash, etc. Additionally, the catheter undergarment device 100 improves comfort and self-confidence for any male or female wearing a catheter.

Further, the catheter undergarment device 100 comprises a body component 102 configured as a diaper or adult undergarment. The body component 102 is constructed using a waterproof material to catch spills, is non-chafing, and fits snugly like regular underwear. The body component 102 comprises a rear portion 112, a front panel 114 extending from the rear portion 112 and opposing side panels 116. The rear portion 112 is adapted to be positioned underneath the buttocks of a user 106, with the opposing side panels 116 being secured around the legs 118 of the user 106 before connecting to the front panel 114 so as to form a unified undergarment configuration. Typically, the rear portion 112 and front panel 114 are integrally formed together. Further, the opposing side panels 116 are releasably secured to each other via Velcro tabs 120, clasps, buttons, bands, adhesives, straps, snaps, etc. The use of side panels 116 adapted to be removably secured together aids in applying or removing the undergarment (i.e., body component 102) on immobile patients, such as those who are bedridden.

Additionally, the upper end 122 of the front panel 114 and the rear portion 112 forms the waistband of the undergarment (i.e., body component 102) and is adapted to encircle the user's waist. The lower end 124 of the front panel 114 and the rear portion 112 includes a curved portion on the sides to accommodate leg openings when the undergarment (i.e. body component 102) is formed.

In one embodiment, the rear portion 112, front panel 114, and opposing side panels 116 are all integrally formed together as one integral piece (i.e., unitary structure).

Furthermore, the body component 102 comprises a flexible, waterproof material. Further, the body component 102 is preferably at least partially comprised of a material and configuration which will act as an absorbent for body fluids and the like, such as those resulting from urination or defecation.

Additionally, the front panel 114 and rear portion 112 both include an outer surface 126 and an inner surface 128. The inner surface 128 contacts a user's skin and is at least partially comprised of an absorptive material for absorbing liquids, feces, and such. The outer surface 126 contacts a user's outer clothing 110 and is waterproof, so as to prevent leaks on the outer clothing 110, etc.

Further, the front panel 114 comprises a catheter opening 104. The catheter opening 104 is a through-hole through the front panel 114 for accepting a catheter tube 130. Specifically, the catheter opening 104 is a through-hole through which a catheter tube 130 may be extended. A tube retainer 132 may also be utilized to guide the catheter tube 130 out of the body component 102 through the catheter opening 104. The catheter opening 104 is adapted to allow a catheter tube 130 or other medical instrument or device to pass through the catheter opening 104. The present invention is preferably utilized with an indwelling catheter which comprises a catheter tube 130 having an end inserted subcutaneously. The distal end of the catheter tube 130 extends through the catheter opening 104 and, in some embodiments, a tube retainer 132 helps to guide the tube 130 through the opening 104. The tube retainer 132 lines the bottom of the opening 104 and may be utilized to guide the catheter tube 130 as it exits the catheter opening 104. The catheter tube 130 may extend through any portion of the catheter opening 104.

In one embodiment, the catheter opening 104 comprises a pad 134 which is adapted to be positioned behind the catheter opening 104 between the body of the user 106 and the undergarment (i.e., body component 102). The pad 134 is generally comprised of a piece of fabric or other absorbent material which is adapted to be positioned behind the catheter opening 104 and around the catheter tube 130. The pad 134 acts to prevent leaks, drips, or spills from the catheter tube 130.

Further, the catheter opening 104 comprises antibiotic ointment 136 around the perimeter of the opening 104. Any suitable antibiotic ointment 136 can be utilized as is known in the art. The antibiotic ointment 136 helps treat or stop any infections from happening.

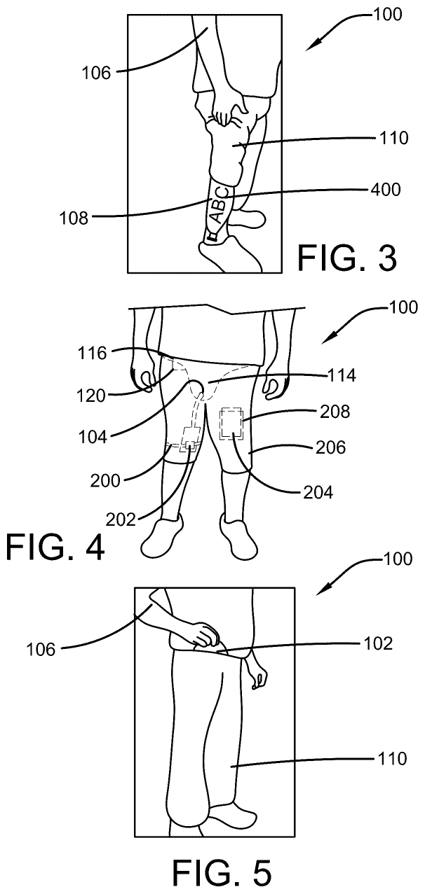

As shown in FIGS. 2-4, the catheter undergarment device 100 comprises leg straps 200 that secure the catheter bag 108 to a user's inner leg 118. The leg straps 200 typically are secured via Velcro 202, snaps, adhesives, buckles, etc. In another embodiment, the catheter undergarment device 100 includes an inner leg pouch 204 on one leg 118 that is secured via leg straps 200 around a user's leg 118. The leg straps 200 typically are secured via Velcro 202, snaps, adhesives, buckles, etc. The tube 130 and catheter bag 108 are hidden within the inner leg pouch 204. The inner leg pouch 204 is constructed to expand and it allows for the catheter bag 108 to expand when retaining urine collection. The leg straps 200 for either embodiment can be knee length, calf length, or ankle length and made of an elastic and expandable material.

In one embodiment, the side panels 116 of the body component 102 can be extended down, creating a leg component 206, such that the body component 102 is configured as a pair of shorts. In this embodiment, the leg component 206 comprises a pocket 208 which can retain the catheter bag 108, such that the catheter bag 108 does not have to be secured with straps 200 to a leg 118. Specifically, leg straps 200 can irritate skin from the leg straps 200 constantly sliding up and down legs 118. To eliminate this movement, users 106 typically would overtighten their leg straps 200, which in turn would reduce blood flood to their leg 118, further complicating an already bad situation. Users 106 also inadvertently dislodge tubes 130 while trying to undress or go to the restroom.

Further, to keep the urine discharge from moving up in the tubing 130, a one-way valve 210 is recommended. The one-way valve 210 can be pressure sensitive and can be positioned in the catheter tube 130 or at the entry into the catheter bag 108.

In yet another embodiment, the catheter undergarment device 100 comprises a plurality of indicia 400. The body component 102 of the device 100 may include advertising, a trademark, or other letters, designs, or characters, printed, painted, stamped, or integrated into the body component 102, or any other indicia 400 as is known in the art. Specifically, any suitable indicia 400 as is known in the art can be included, such as, but not limited to, patterns, logos, emblems, images, symbols, designs, letters, words, characters, animals, advertisements, brands, etc., that may or may not be undergarment, catheter, or brand related.

As shown in FIG. 5, the catheter undergarment device 100 is manufactured from waterproof material to catch spills, is non-chafing, and fits snugly like regular underwear, or other suitable materials as is known in the art, or a combination of such materials. Further, the device 100 is manufactured of a stretchable, elastic-type fabric that may include fabric blends such as cotton/poly/Spandexe or Nylon® Tactel® & LYCRA®, etc.

In one embodiment, the catheter undergarment device 100 is manufactured of cotton fabric, which is selected as a textile substrate because of its wide use in hospital and clinical settings. The catheter undergarment device 100 can be cut in the dimensions of 5 cm×3 cm, washed to remove any adhered impurities and then dried at ambient conditions and stored at 4° C.

In another embodiment, the catheter undergarment device 100 is manufacture using a layer-by-layer deposition of AgNPs on surgical fabric. Silver nanoparticles solution (50 ml of 1 mM) were made and utilized as anionic solution in the current method. A layer-by-layer assemblage was set-up where a fabric swatch dipped in PDAC solution for 10 min followed by 2 successive washing steps in distilled water, each for 5 min. The fabric was then dipped in AgNPs solution for 10 min followed by 2 further washing steps. This method leads to the formulation of a single layer in a period of 40 min. The whole procedure was repeated to produce samples with 5, 10, 25, 15 and 20 layers. At room temperature, the fabric swatches were dried for 3-4 h and stored in a moisture free environment.

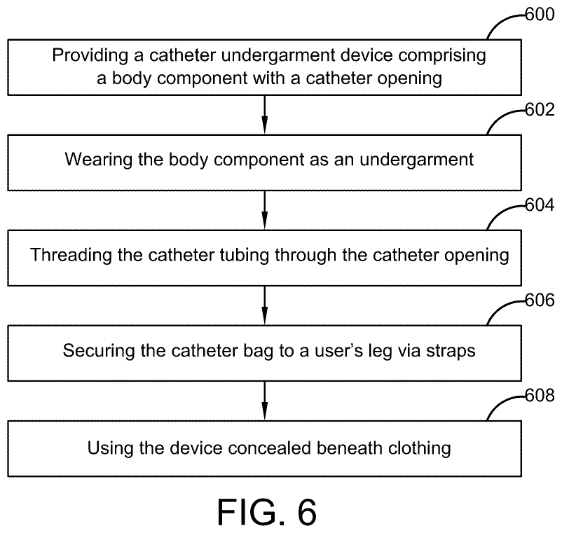

FIG. 6 illustrates a flowchart of the method of allowing users to discreetly hide their catheter bags without worry of spills. The method includes the steps of at 600, providing a catheter undergarment device comprising a body component with a catheter opening. The method also comprises at 602, wearing the body component as an undergarment. Further, the method comprises at 604, threading the catheter tubing through the catheter opening. The method also comprises at 606, securing the catheter bag to a user's leg via straps. Finally, the method comprises at 608, using the device concealed beneath clothing.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “catheter undergarment device”, “catheter device”, “undergarment device”, and “device” are interchangeable and refer to the catheter undergarment device 100 of the present invention.

Notwithstanding the foregoing, the catheter undergarment device 100 of the present invention can be of any suitable size and configuration as is known in the art without affecting the overall concept of the invention, provided that it accomplishes the above-stated objectives. One of ordinary skill in the art will appreciate that the catheter undergarment device 100 as shown in FIGS. 1-6 is for illustrative purposes only, and that many other sizes and shapes of the catheter undergarment device 100 are well within the scope of the present disclosure. Although the dimensions of the catheter undergarment device 100 are important design parameters for user convenience, the catheter undergarment device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A catheter undergarment device that provides a user with a means for concealing catheter bags, the catheter undergarment device comprising:
a body component; and
a pair of leg straps;
wherein the body component includes a catheter opening;
wherein a catheter tube protrudes from the catheter opening;
wherein the pair of leg straps secure a catheter bag to a user's inner leg; and
further wherein users can wear a variety of clothing while concealing the catheter tube and catheter bag.

2. The catheter undergarment device of claim 1, wherein the body component is configured as a diaper or adult undergarment.

3. The catheter undergarment device of claim 2, wherein the body component comprises a rear portion, a front panel extending from the rear portion and opposing side panels.

4. The catheter undergarment device of claim 3, wherein the rear portion is adapted to be positioned underneath buttocks of a user, with the opposing side panels being secured around legs of the user before connecting to the front panel so as to form a unified undergarment configuration.

5. The catheter undergarment device of claim 4, wherein the rear portion and the front panel are integrally formed together.

6. The catheter undergarment device of claim 5, wherein the opposing side panels are releasably secured to each other via hook and loop tabs.

7. The catheter undergarment device of claim 6, wherein upper end of the front panel and the rear portion forms a waistband of the body component and is adapted to encircle a user's waist.

8. The catheter undergarment device of claim 7, wherein a lower end of the front panel and the rear portion includes a curved portion on sides to accommodate leg openings when the body component is formed.

9. The catheter undergarment device of claim 8, wherein the front panel and rear portion both include an outer surface and an inner surface, such that the inner surface contacts a user's skin and is at least partially comprised of an absorptive material for absorbing liquids, and the outer surface contacts a user's outer clothing and is waterproof to prevent leaks.

10. The catheter undergarment device of claim 9, wherein the catheter opening is a through-hole through the front panel for accepting a catheter tube.

11. The catheter undergarment device of claim 10, wherein the catheter opening comprises antibiotic ointment around a perimeter of the catheter opening.

12. The catheter undergarment device of claim 11, wherein the pair of leg straps are secured via hook and loop fasteners.

13. The catheter undergarment device of claim 12, wherein the pair of leg straps can be knee length, calf length or ankle length and made of an elastic and expandable material.

14. A catheter undergarment device that provides a user with a means for concealing catheter bags, the catheter undergarment device comprising:
a body component comprising a rear portion, a front panel extending from the rear portion and opposing side panels; and
a pair of leg straps secured around a user's inner leg via hook and loop fasteners;
wherein the rear portion is adapted to be positioned underneath buttocks of a user, with the opposing side panels being secured around legs of the user before connecting to the front panel so as to form a unified undergarment configuration;
wherein the rear portion and the front panel are integrally formed together;
wherein the opposing side panels are releasably secured to each other via hook and loop tabs;
wherein upper end of the front panel and the rear portion forms a waistband of the body component and is adapted to encircle a user's waist;
wherein a lower end of the front panel and the rear portion includes a curved portion on sides to accommodate leg openings when the body component is formed;
wherein the front panel and rear portion both include an outer surface and an inner surface, such that the inner surface contacts a user's skin and is at least partially comprised of an absorptive material for absorbing liquids, and the outer surface contacts a user's outer clothing and is waterproof to prevent leaks;
wherein the body component includes a catheter opening which is a through-hole through the front panel for accepting a catheter tube;
wherein the catheter opening comprises antibiotic ointment around a perimeter of the catheter opening;
wherein the pair of leg straps can be knee length, calf length or ankle length and made of an elastic and expandable material; and
further wherein users can wear a variety of clothing while concealing the catheter tube and catheter bag.

15. The catheter undergarment device of claim 14, wherein a catheter bag is concealed via an inner leg pouch secured to one leg via a pair of straps with hook and loop fasteners.

16. The catheter undergarment device of claim 14, wherein the side panels of the body component can be extended down creating a leg component with a pocket for retaining the catheter bag.

17. The catheter undergarment device of claim 14, wherein the rear portion, front panel, and opposing side panels are all integrally formed together as one integral piece.

18. The catheter undergarment device of claim 14, wherein the catheter opening comprises a pad to prevent leaks, which is adapted to be positioned behind the catheter opening between body of the user and the body component.

19. The catheter undergarment device of claim 14 further comprising a plurality of indicia.

20. A method of allowing users to discreetly hide their catheter bags without worry of spills, the method comprising the following steps:
providing a catheter undergarment device comprising a body component with a catheter opening;
wearing the body component as an undergarment;
threading the catheter tubing through the catheter opening;
securing the catheter bag to a user's leg via straps; and
using the device concealed beneath clothing.
